(19) United States
(12) Patent Application Publication
(10) Pub. No.: US 2023/0148760 A1
(43) Pub. Date: May 18, 2023

(54) DETECTION APPARATUS FOR BEDDING

(71) Applicant: WITHINGS, ISSY LES MOULINEAUX (FR)

(72) Inventors: Samuel CLERC, ISSY LES MOULINEAUX (FR); Vincent VERCAMER, ISSY LES MOULINEAUX (FR); Pierre-Antoine CUNIASSE, ISSY LES MOULINEAUX (FR)

(21) Appl. No.: 17/960,525

(22) Filed: Oct. 5, 2022

(30) Foreign Application Priority Data
Oct. 28, 2021 (FR) 2111494

(51) Int. Cl. A47C 23/06; A47C 31/12

(52) U.S. Cl. CPC A47C 23/062; A47C 31/123; A61B 5/4806

(57) ABSTRACT

An acquisition apparatus for acquiring physiological data of an individual, configured to be installed in a bed frame, with an acquisition portion which includes a sensor intended to capture mechanical waves transiting in the bed frame, a support device including one or more rigid panels, the support device being configured to rest on bed frame elements which may be discontinuous for example slats, so that the support device is interposed between the bed frame elements and the sensor, and may provide a substantially continuous support to the sensor receiving the mechanical waves generated by the individual through the bedding, if necessary with folding zones between panels.

### CROSS-REFERENCE TO RELATED APPLICATIONS

This application claims priority to French Patent Application No. 2111494, filed Oct. 28, 2021, the entire content of which is incorporated herein by reference in its entirety.

### FIELD

The invention relates to systems and apparatus that can be placed in bedding to collect sleep data from the user(s) of the bedding in question.

### BACKGROUND

Document EP2873368 describes a device to be put under or on the mattress, in order to detect by ballistocardiography at least the movements, the cardiac and respiratory rhythms (by detection of the movements or even micro-movements) of an individual resting on the mattress, in order to monitor the sleep of the individual.

Document EP3508187 describes a similar device, with an inflatable pocket, which aims to mitigate difficulties of sensitivity related to the rigidity of the mattress. In order to alleviate this difficulty, it has been proposed in particular to put a rigid structure distinct from the mattress to distribute the variable forces on the device. This structure is placed between the mattress and the inflatable bag. Such a structure is not convenient to use and decreases the manipulability of the device (packing, unpacking, storage, etc.).

### SUMMARY

Nevertheless, the inventors realized that prior art devices could present performances that still depended on the type of bedding. Indeed, they discovered that the nature of the bed frame, on which the device rests, could impact the performances.

It is thus proposed an apparatus for acquiring physiological data from a living being (generally a human) with a lower sensitivity to the bed frame.

According to one aspect, there is provided herein an acquisition apparatus for acquiring physiological data from a living being, the acquisition apparatus being configured to be installed in a bedding, the acquisition apparatus comprising an acquisition portion that comprises:
a sensor designed to capture mechanical waves passing through the bedding (typically generated by the living being),
a support device comprising at least one rigid panel, positioned to support the sensor.

The support device is configured to, in a configuration of use of the acquisition apparatus in the bedding, rest on bed frame elements such that the support device is interposed between the bed frame elements and the sensor. The bed frame elements may be discontinuous, such as slats.

This provides a substantially continuous support for the sensor receiving mechanical waves generated by a user through the bedding. The mechanical waves come from the user’s heartbeat, breathing or other body movements.

Thanks to these arrangements, it is avoided that the sensor goes down between the slats and gets stuck there. It is also avoided that the sensor collapses locally and loses sensitivity in detecting mechanical waves (especially the weaker ones of the heart and the breathing).

The sensor may be of various technologies, namely a motion and/or pressure sensor, such as a pneumatic, piezoelectric or strain sensor, such as a strain gauge, or other technology. The support device, placed under the sensor, provides an adequate support to maximize the efficiency, sensitivity and accuracy of the sensor.

In the expression ‘rigid panel’, it is understood by ‘rigid’ that for example the structure of which the panel is composed has a rigidity equivalent to a material with a Young’s modulus greater than 1 GPa (and for example less than 20 GPa, for example less than 10 GPa, for example less than 5 GPa) It should be noted that the panel(s) does however have some flexibility to conform to the general geometry of the slats of the bed frame, which themselves have a flexibility in reaction to a weight (i.e., to an individual resting on the mattress).

It should be noted that the thickness of the acquisition portion is small, for example less than 10 mm or even less than 5 mm. The acquisition portion of the apparatus can thus be easily inserted and interposed between the mattress and the bed frame, without substantially protruding into the mattress, nor damaging or permanently deforming the mattress.

It should also be noted that the acquisition apparatus works properly with a mattress of any thickness on top.

The acquisition apparatus may integrate an electronic unit, to format and/or process raw data delivered by the sensor. This is done without substantial additional thickness.

According to one option, the acquisition portion extends along a longitudinal direction (X) and the support device comprises a plurality of rigid panels arranged next to each other along the longitudinal axis of the acquisition portion. Two adjacent panels may be connected to each other by a folding zone allowing folding of the support device. In an embodiment, the folding is along an axis parallel to a direction orthogonal to the longitudinal direction (X) (called the transverse direction (Y)).

Therefore, the folding zones allow the acquisition portion and the support device to be folded for storage of the acquisition apparatus. The folding zones also allow the acquisition portion to conform to the geometry of the bed frame, and to follow the elasticity of the slats, knowing that the slats do not bend uniformly, e.g. according to the distribution of the weight applied on the mattress.

The term ‘next to’ is to be understood here as adjacent or slightly apart (e.g. less than 3 cm). In other words, the panels follow each other along the longitudinal axis, touching or being separated by a space.

It should be noted that instead of the term ‘panel’, the term ‘plate’ or ‘board’ could be used to designate the elements of the supporting device. The panel may be solid or apertured (e.g., latticework).

According to one option, each of the folding zones may be formed as a hinge zone, for example parallel to the transverse direction (Y). In this way, a compact folded form with several hinge zones may be obtained, either in the form of a roll or in the form of an accordion.

According to one option, the panels are integrated into a sheath, such as a flexible sheath, that extends along the longitudinal direction (X), the zones of the flexible sheath that are free of panels may form folding zones. The flexible sheath is a simple means of holding the panels in place relative to each other and to the sensor. “Holding in place” must be understood outside of the bending movement. The flexible sheath may be made of a continuous or discontinuous material, e.g., apertured like a net.

According to one option, the flexible sheath forms a plurality of housings, with the panels inserted into the housings, for example one panel per housing. The housings are arranged consecutively along the longitudinal direction (X). Between two housings there is a connecting zone, which forms a folding zone as described above. This forms a simple and easy to implement solution for assembling the panels in the flexible sheath. A slightly deformable opening may be provided in the fabric of the sheath to allow the insertion of the rigid panel, after what it is retained inside the housing naturally by the elasticity of the fabric. Alternatively, the sheath is sewn or sealed so that the panels are no longer accessible. The sheath prevents the user from being in contact with the rigid panels (risk of cut or other). It also provides a more comfortable touch.

According to one option, each of the panels has a generally rectangular shape and has a constant thickness, for example less than 3 mm or even 2 mm or even 1 mm. The thickness may depend on the material. Each panel is lightweight and inexpensive to manufacture.

According to one option, there may be multiple panels in a housing. This further increases the possibilities of rolling or folding to turn the acquisition apparatus into a space saving item.

According to one option, the sensor has a sensor width (LY) along a transverse direction (Y) and each rigid panel extends along the transverse direction over substantially the entire sensor width. Substantially means at least 80% or even 90% of the sensor width of the sensor.

According to one option, the acquisition apparatus may further comprise a cover enveloping at least the acquisition portion. The cover thereby protects the sensor.

According to one option, the cover is removable. This allows the cover to be washed or cleaned if necessary.

According to one option, the cover is separate from the flexible sheath. The flexible sheath holds the rigid panel assembly together, and the cover covers the assembly which also includes the sensor.

According to one option, a portion of the cover acts as a flexible sheath. In other words, the flexible sheath as described above is part of the cover that protects the sensor. Thus, a single piece of fabric equipped with seams acts as the flexible sheath and the cover enveloping the acquisition portion. In other words, the cover and the sheath are embodied by a single piece, for example of fabric, which performs both functions, i.e., the placement and holding of the panels as well as the enveloping protection. By integrating the rigid panel or rigid plates into the cover, a simple to use acquisition apparatus is obtained, formed of few distinct parts.

In other words, the cover may comprise a top side, intended to be against the mattress and a bottom side, intended to be against the bed frame elements, the cover being configured to receive the sensor between the top side and the bottom side, and wherein the flexible sheath forms the bottom side of the cover.

According to one option, the acquisition apparatus may further comprise an attachment system (or at least one attachment). The attachment system may extend, directly or indirectly (e.g., from the cover) from the acquisition portion, on either side of the acquisition portion, in particular along the transverse Y direction.

It is noted that the attachment system may be in the form of straps equipped with Velcro™ hook and loop surface to secure or attach the acquisition apparatus.

This allows the acquisition apparatus to be secured to one or more slats. This ensures that the acquisition apparatus remains in place even with a substantial inclination of the bed frame (medical bed or more generally reclining bed).

According to one option, it may be provided that the attachment system is detachably attached to the acquisition apparatus, in particular to the cover. In this way, there is some freedom to position the attachment system in relation to the acquisition portion and the slats of the bed frame.

According to one option, the sensor may include a pneumatic chamber. This is a well-controlled, reliable and durable solution.

The air chamber may be equipped with bridges. Such bridges limit the bulging of the air chamber when it is inflated.

According to one option, an electronic unit may be additionally provided, which in particular allows the processing of raw data delivered by the sensor. The electronic unit may include, for example, a transducer, a signal conditioner, or a wireless communication coupling module. The electronic unit may be mounted in a case, located at one longitudinal end of the acquisition apparatus. The case and the sensor are then in close proximity to each other along the longitudinal direction (X).

According to one option, the electronic unit may be powered by a local electrical battery. According to another option, the acquisition apparatus may comprise a connection cable to be powered from an external power supply.

According to one option, the sensor is a pneumatic sensor, which may be coupled to a pump and a discharge valve. The pneumatic chamber is inflated for the acquisition phases and may be deflated for the storage phases. The pump and the discharge valve may be mounted in a case, for example the case housing the electronic unit.

According to one option, the acquisition apparatus comprises a visual marker that indicates to the user on which sides the acquisition apparatus is to be installed. In particular, the visual marker and the support device may each be on opposite sides of the sensor. In this way, when the user installs the acquisition apparatus, it is enough for him to keep the visual marker in sight so that the acquisition apparatus is naturally put on the right side (i.e., the support device between the bed frame elements and the sensor). The visual marker may be a logo, a shape, the position of the case (e.g., relative to a sensor web), etc. The visual marking may indicate which side of the acquisition apparatus should be against the mattress, thus the support device is on the other side.

According to one option, the acquisition apparatus may be configured to be folded into a storage configuration of the acquisition apparatus. In this way, the acquisition apparatus may assume a shape with a small overall footprint to facilitate delivery, storage, or stowage.

Also provided is a kit comprising at least all of the elements described above for assembling an acquisition apparatus as described above. In particular, the sensor and the support device may be delivered in separate boxes or may be arranged side by side in a box. The user then arranges the elements correctly. When the acquisition apparatus comprises a removable cover which does not integrate the support device, the sensor and the support device may be, at the delivery, outside the cover.

Also proposed herein is an assembly comprising a bed frame element and an acquisition apparatus as described above, wherein the bed frame element comprises two slats having a length (L7) extending along the longitudinal direction (X) and spaced apart from each other by an inter-slat distance (L3) along the transverse direction (Y) and wherein the width (LY) of the sensor is greater than the inter-slat distance and/or each rigid panel extends along the transverse direction (Y) over a width greater than the inter-slat distance, for example greater than the inter-slat distance and the width of one slat (or even two slats).

In other words, herein there is proposed an acquisition apparatus for acquiring physiological data of a living being, configured to be installed in a bedding, the acquisition apparatus comprising:
an acquisition portion with a sensor intended to capture mechanical waves transiting in a bedding,
a support device comprising at least one rigid panel, configured to support the sensor,
a cover, configured to enclose at least the acquisition portion.

The support device is configured to, in a configuration of use of the acquisition apparatus in the bedding, rest on bed frame elements such that the support element is between the bed frame elements and the sensor. The bed frame elements may be discontinuous, such as slats.

The cover comprises an upper face, intended to be against the mattress and a lower face, intended to be against the bed frame elements, the sensor being receivable inside the cover between the upper face and the lower face. In particular, the lower face may form a sheath within which the at least one rigid panel is located. The sheath may comprise two substantially mutually parallel sides, which define between them a housing inside which the at least one rigid panel is located. Alternatively, the bottom face forms a sheath that comprises a plurality of separate housings, each housing comprising a rigid panel.

The options mentioned above are generally applicable for this formulation.

According to a particular aspect, it is proposed the acquisition apparatus without the aforementioned support device but with the attachment or attachments. In other words, it is proposed an acquisition apparatus for acquiring physiological data of a living being, configured to be installed in a bedding, the acquisition apparatus comprising an acquisition portion which comprises a sensor for sensing mechanical waves transiting in the bedding, and attachment means which may extend, directly or indirectly (for example from the cover) from the acquisition portion, on either side of the acquisition portion, in particular in the transverse direction (Y) for attaching the acquisition portion to the bed frame elements.

### DESCRIPTION OF THE FIGURES

Other features, details and benefits of the invention will become apparent from the detailed description below, and from an analysis of the appended drawings.

FIG. 1 shows a top view of an acquisition apparatus installed on a slatted bed frame, without a mattress.

FIG. 2 schematically illustrates part of the acquisition portion of the acquisition apparatus, with several rigid panels, and folding zones between panels.

FIG. 3 shows a vertical cross-section along the longitudinal direction of the acquisition portion, an example of an acquisition apparatus with slats.

FIG. 4 shows a schematic view of a six-panel configuration with identical dimensions.

FIG. 5 illustrates schematically in top view a seven-panel configuration of progressively smaller width from one longitudinal end to the other.

FIG. 6 illustrates schematically in top view a nine-panel configuration with increasing widths from the middle to the longitudinal ends.

FIG. 7 represents, in vertical section along the longitudinal direction of the acquisition portion, another example of the acquisition apparatus, a rigid panel not having been shown.

FIG. 8 represents, in vertical section along the longitudinal direction of the acquisition portion, another example of the acquisition apparatus, a rigid panel not having been shown.

FIG. 9 represents, in vertical section along the longitudinal direction of the acquisition portion, another example of the acquisition apparatus, a rigid panel not having been shown.

FIG. 10 shows, in perspective, a set of panels hinged to each other.

FIG. 11 represents, in vertical section along the longitudinal direction of the acquisition portion, another example of the acquisition apparatus, a rigid panel not having been shown.

FIG. 12 shows a general perspective view of an example of an acquisition apparatus.

FIG. 13 shows, in vertical section, only the cover in an embodiment shown in FIG. 9.

### DETAILED DESCRIPTION

In the various figures, the same references designate identical or similar elements. For reasons of clarity, some elements are not necessarily shown to scale. In particular, to facilitate understanding, the thicknesses and spacings along the Z axis have been artificially increased in FIGS. 7, 8, 9 and 11.

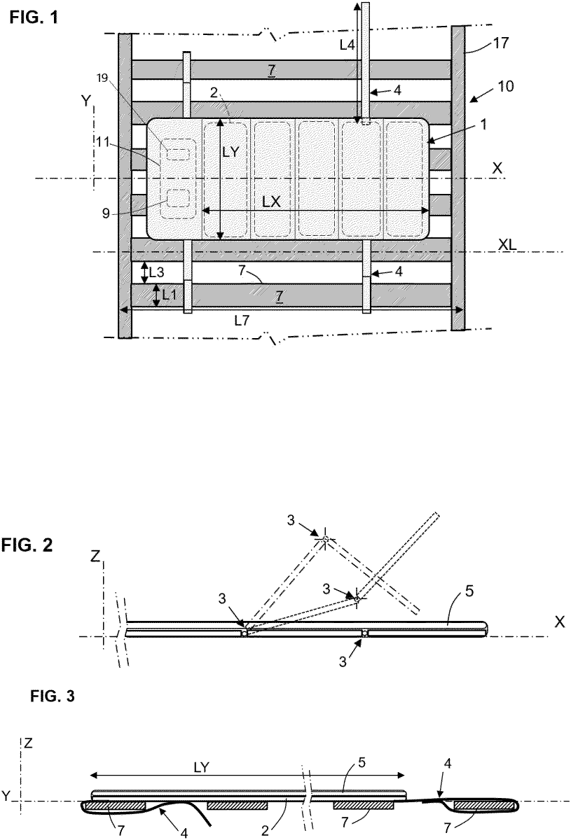

In FIG. 1, a bed frame 10 is shown in part, which is a slatted bed frame that comprises two side rails 17 and slats 7 transverse to the side rails 17, the slats 7 being supported at least at both ends by the side rails 17. The dimension called “width” of the bed or width of the bed frame, marked L7, may be 90 cm, 120 cm, 140 cm, 160 cm. Other dimensions are not excluded. It will be appreciated that two bed frames may be placed side by side for large bed widths.

A bed frame element 10 is used to designate the slats or any other part of a bed frame on which the mattress rests.

According to a direction called main direction of the bed (called length), the spacing between the slats, called inter-slat distance, is L3 and the width of the slats is L1.

An acquisition apparatus 1 is installed on the slats. A mattress 90 (shown in mixed lines only in FIG. 9) is then installed on top of the acquisition apparatus 1. It should be noted that the characteristics of the mattress do not matter, for example in terms of its thickness or flexibility. The acquisition apparatus may work for any type of mattress.

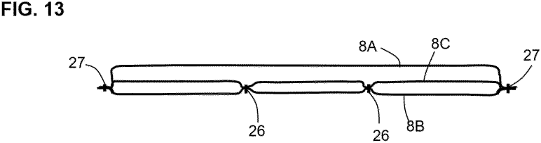

FIG. 12 shows a perspective view of an acquisition apparatus according to an example of an embodiment.

It should be noted that in two-people wide bed, two acquisition apparatus may be installed, for example one for each occupant.

The bed may be a conventional domestic bed. However, the invention also relates to beds of the medical bed type. In both cases, a partial tilting function of the bed frame element may be provided.

In the following, a longitudinal direction X is defined, which is defined in relation to the length of the acquisition apparatus 1 while a transverse direction Y is defined in relation to the width of the acquisition apparatus. The longitudinal X and transverse Y directions are typically orthogonal to each other. This XY reference frame is therefore different from the usual directions relating to the bedding itself.

The acquisition apparatus 1 comprises an acquisition portion 1A adapted to capture the mechanical waves generated by a living being positioned on the mattress, as discussed elsewhere. The acquisition portion is presented as a thin sheet. In addition, the acquisition apparatus 1 may include auxiliary elements, such as an electronic unit 9 and/or a pneumatic unit, which will be discussed later. The auxiliary elements may be housed in a case 11 attached, on one face of the thin sheet, to a longitudinal end of the sheet.

The user generates mechanical biological signals by the movement of his internal organs or by a body movement, voluntary or reflex. Breathing-induced movements are part of these movements, including the mechanical manifestation of snoring. The movements induced by the heartbeat are part of these movements. These waves pass through the mattress 90, whatever its thickness or consistency, and are received by the acquisition portion 1A, in particular the sensor 5.

The acquisition portion 1A is thin and is, in normal use, interposed between the slats and the mattress. The acquisition portion comprises a sensor 5. The sensor may be a motion sensor or a pressure sensor (one not necessarily excluding the other). For example, the sensor may be a pneumatic sensor or a piezoelectric sensor, a deformation sensor (deformation gauge for example). However, other technologies are not excluded. The sensor 5 is arranged in the acquisition portion 1A of the acquisition apparatus 1.

In addition to the sensor 5, the acquisition portion 1A comprises a support device SP, intended to be placed in normal use between the sensor 5 and the bed frame element 10. The support device SP comes directly or indirectly into contact with the sensor 5 to serve as a support for it.

When the acquisition apparatus 1 is set up, each slat 7 extends along an axis XL parallel to the longitudinal axis X of the acquisition apparatus (i.e., of the acquisition portion). The transverse direction Y of the acquisition portion corresponds to the usual longitudinal direction of the bed.

The slats may be single or double, i.e., two slats superimposed.

The slats may be flat or curved. Often, each slat has a curvature that forms a slight hump in the middle of the bed frame. This curvature is cancelled or reversed by a load applied to the slat.

Each slat has some flexibility to contribute to the comfort of the user using the bed. Each slat 7 deforms elastically substantially in proportion to the weight that is applied against it.

The distance L3 between the slats may be of the same order as the width of the slats L1. In some configurations, L3 is greater or less than L1. The spacing of the slats may be constant or not on the whole bed frame.

The width of the L1 slats is in practice between 5 cm and 15 cm, most often around 10 cm.

The dimension of the sensor 5 in the transverse direction Y (also called the width of the sensor) is noted LY and the dimension of the sensor in the longitudinal direction X is noted LX.

LY is dimensioned so that it is clearly greater than L1 or L3. For example, the condition LY > 2 × L1, or LY > 2 × L1 + L3, or LY > 2× (L1 + L3) or LY > 3 × L1 or LY > 3× (L1 + L3) may be chosen.

In other words, generally, the sensor 5 is installed astride at least two slats 7. In some cases, it may rest on three or more slats.

The width LY of the sensor 5 may be between 10 cm and 50 cm.

Generally, the length LX of the sensor 5 may be between 40 cm and 60 cm.

The length L7 of the slats may be between 80 cm and 120 cm.

The support device SP comprises one or more rigid panels 2. In the illustrated examples, the number of rigid panels 2 is between 6 and 10; however, any number falls within the scope of the invention without excluding a single rigid panel configuration. There may be between 3 and 20 panels, or even between 5 and 15 panels. The rigid panels 2 may be arranged adjacently along the longitudinal direction X.

In a configuration of normal use of the acquisition apparatus in the bedding, the acquisition portion 1A comes to rest on the slats or more generally on the bed frame elements that may be discontinuous. At least 80% of the surface of the sensor 5 is arranged on the support device SP, and in an embodiment at least 90%.

The support device SP is configured, in use, to be interposed between the bed frame elements 10 and the sensor 5. It thus provides, even in the case of discontinuous bed frame elements, a substantially continuous support to the sensor receiving the mechanical waves generated through the bedding by a user lying on the mattress. The sensitivity of the sensor to mechanical waves is thus improved and no longer depends on the type of bed frame element.

Having several rigid panels 2 allows for a foldable support device SP (for storage or packaging). In this regard, the support device SP may provide a folding zone 3 between two adjacent panels. The folding zone 3 may allow a folding with an axis parallel to the transverse direction Y. In the case where several panels 2 make up the support device SP, a folding zone 3 is provided between pairs of adjacent panels 2a, 2b (see FIGS. 7, 8 or 10), a folding zone 3 allowing a folding along the axis parallel to the transverse direction Y.

According to an example, when the support device comprises N panels, there are provided N-1 folding zones, the foldings may be substantially parallel to each other (i.e. there is less than 5 degree difference between the corresponding longitudinal axes of the folding zones).

The folding direction may be the same for all folding zones or alternatively in the manner of an accordion, both possibilities being illustrated in FIG. 2. The possible folding amplitude of each folding zone may be symmetrical or not.

In an embodiment, illustrated in FIG. 10, the support device comprises a plurality of contiguous panels 2a, 2b interconnected by a mechanical hinge (or articulation) acting as a folding zone 3. The articulation may be along the axis X3 which is parallel to the transverse direction Y.

In an embodiment illustrated in FIGS. 7, 8 and 9, the panel(s) 2 are integrated into a flexible sheath 6 (or envelope). For example, the panels may be arranged from one another along the longitudinal direction X. The panels may be contiguous as described above or adjacent but not contiguous. The areas of the flexible sheath without panels may then serve as folding zones 3. In particular, the flexible sheath 6 comprises a top side and a bottom side.

The flexible sheath may be made of woven or non-woven fabric. The use of a polymeric plastic film may be considered. The use of a fabric based on biodegradable natural fibers is also considered. The sheath 6 may be a continuous fabric or a mesh fabric or a net. Locally, the thickness of the sheath is very small (e.g. less than 3 mm), the fabric or equivalent has an important flexibility to bending; there may also be some longitudinal elasticity of the material.

The flexible sheath 6 may form a single housing that receives the panel or the plurality of panels. Alternatively, the flexible sheath 6 comprises a plurality of housings 24, typically separated by a junction zone 26. The panels are inserted into the housings. According to one configuration, there is one panel per housing. The housings 24 may be arranged next to each other along the longitudinal direction (X), separated from each other by at least one joining zone 26, or even by a joining zone 26 between each pair of adjacent panels 2. The joining zone 26 acts as a folding zone 3, which may be orthogonal to the longitudinal direction.

In order to create the joining zone 26, the top side and the bottom side of the flexible sheath 6 may be joined locally to thereby form the housing. Specifically, a seam connects the top side of the sheath to the bottom side of the sheath in the joining zone 26. This ensures a spacing between the two adjacent panels that allows for a large folding amplitude, or even almost an overlap of two adjacent panels (for example, greater than 160°).

In some embodiments, the acquisition apparatus 1 may further comprise a cover 8 enveloping at least the acquisition portion 1A.

Said cover 8 may also perform the flexible sheath function or may be completely separate from the independent flexible sheath, as shown in FIG. 8. The sheath is then housed inside the cover, so as to be positioned between the bed frame elements and the sensor.

In a configuration where the cover acts as a sheath, as illustrated in FIGS. 9 and 13, the cover 8 comprises an upper face 8A, intended to be against the mattress and a lower face 8B, 8C, intended to be against the bed frame elements. The sensor is then received inside the cover between the upper face and the lower face.

In one embodiment, the bottom side comprises the sheath as described above.

For this purpose, a piece of fabric is arranged in three sections on a surface corresponding at least to the surface of the sensor.

The upper panel 8A is intended to cover at least the sensor 5. The lower panel 8B is intended to be interposed between the panels of the support device SP and the bed frame elements. The intermediate panel 8C is interposed between the sensor 5 and the panels of the support device. Connecting seams 27 may be made on the outer edge. Intermediate seams 26 may be made to delimit and form housing for the panels 2.

In an alternative configuration illustrated in FIG. 11, there may be several panels or plates in each housing. The relative position of the plates in the same housing may be changed, for example, for a complete snail-like winding of the acquisition portion.

Each panel has in the examples a general rectangular shape, with a small thickness (for example between 1 and 3 mm).

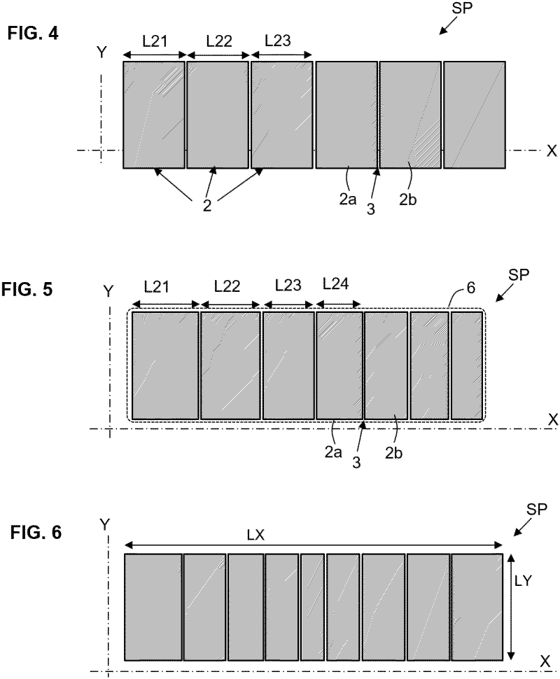

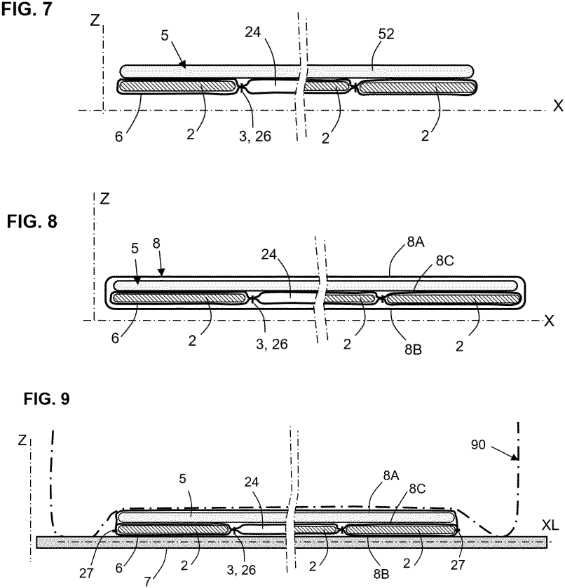

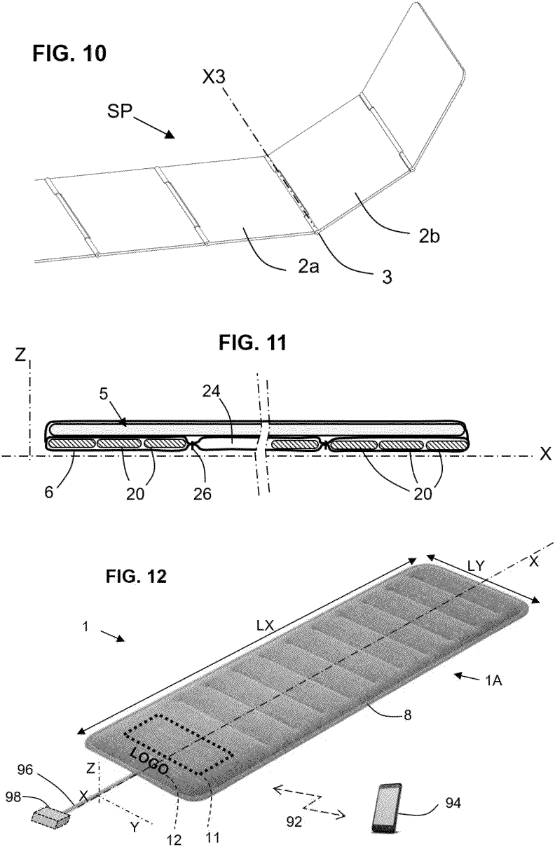

Regarding the dimension of each panel along the longitudinal axis X, according to an example of embodiment shown in FIG. 4, all panels may have the same dimension L21=L22=L23 as shown in FIG. 4. The width of the panels L2i=L21 may be between 3 cm and 10 cm. It may be chosen L2i = 4 cm or L2i = 5 cm.

The dimensions of the panels along the transverse axis Y are substantially equivalent to the width of the sensor LY, so that substantially the entire sensor rests on the support device. By substantially equivalent or substantially, it is meant that said dimension of the panels is equal to at least 80%, or even 90% or 95%, of the width of the sensor LY.

According to another example shown in FIG. 5, the width L2i is increasingly smaller from one longitudinal end to the other: L21>L22>L23 etc....

According to another example shown in FIG. 6, the width L2i is minimal in the middle and the panels are increasingly wider towards the ends.

Any type of plastic material may be used to make the rigid panels, for example PVC, polyethylene, polypropylene, ABS. The use of natural material such as wood is also possible.

To place the acquisition apparatus 1, the user may be guided by one or more visual markers 12 (see ‘LOGO’ on FIG. 12) arranged on the acquisition apparatus 1. Typically, in the case of a cover, the marker(s) are arranged on the cover. The marker 12 may be a brand, a logo, or any sign visually differentiating the two sides of the acquisition apparatus, such as the position of the case in relation to the sensor sheet or the seams on the cover. Typically, the visual marker 12 and the support device SP are positioned on either side of the sensor 5, so that if the user maintains visual contact with the marker 12 during installation, the user is assured that the acquisition apparatus 1 is positioned on the correct side.

As illustrated in FIGS. 1 and 3, the acquisition apparatus 1 may further comprise at least one attachment 4 (hereinafter referred to as an attachment system) extending from the acquisition portion on either side of the acquisition portion. In the illustrated example, this attachment system 4 is in the form of retention straps. In an embodiment, there can be at least two attachments. In another embodiment, there can be at least four attachments.

In an embodiment, the acquisition apparatus 1 does not comprise the support device but comprises the attachment 4.

In FIG. 1, the strap shown in the upper right is not attached and is shown in a straight configuration. In the example shown, the acquisition apparatus is equipped with four straps, but a two-strap or three-strap configuration is also possible.

In the illustrated example, the attachments system is formed as fabric straps or fabric bands. The material may be a polymeric plastic or a synthetic or natural fiber fabric.

The length of these strips L4 is sufficient to go around a batten or even around a batten and twice the width of the gap L3 between the battens.

In one example, the length of the strips L4 is between 30 cm and 60 cm.

In the illustrated example, an area of Velcro™ hooks is provided on one part of the strap. On another part of the strap, an area of Velcro™ is provided so that the strap may be attached to itself and its course allows for the winding of at least one batten 7. In other words, in the general case, the strap loops around a slat and returns to attach to itself.

With regard to the possible routes of the strap, with reference to FIG. 3, the end of the strap may be connected to an intermediate portion of the strap (see right-hand part of FIG. 3). In a variant, an end zone of the strap is connected to the cover (see left-hand part of FIG. 3).

According to an option, each of the straps 4 is sewn to the cover 8.

According to another option, each of the straps is detachably associated with the cover 8. For example, a Velcro™ type system may also be used here to attach the strap to an edge area of the cover.

According to an option, not shown in the figures, a longer strap may be provided, which extends on both sides of the acquisition portion with a length close to 2×L3 + LY; whereby a single strap may make a looped path on both sides of the acquisition portion. With two long straps, four attachment zones are provided on the slats. It will be appreciated that the straps will be folded on the acquisition portion when the acquisition apparatus is to be stored.

In an alternative embodiment not shown in the figures, laces may be provided as attachments means.

In an alternative embodiment not shown in the figures, a locking buckle of the type used for clothing belts may be provided on each strap.

As mentioned previously, the acquisition apparatus may also include an electronic unit 9, which may be housed in a case 11. In this electronic unit, an electronic circuit for formatting and/or processing the raw data delivered by the sensor may be provided. A wireless communication coupler may also be provided. The acquisition apparatus may also include a local power supply battery.

In the case where the sensor is a pneumatic sensor, the sensor is in the form of an inflatable pneumatic chamber. Then the acquisition apparatus may further include a miniature pneumatic pump 19 to inflate the pneumatic chamber, and a discharge valve to bleed the pneumatic chamber. The pump 19 may be housed in the case 11 as well.

As visible in FIG. 12, the acquisition apparatus may include a cable 96, particularly for powering the acquisition apparatus. The end of the cable is equipped with a connector 98 (e.g., micro-USB).

Concerning data transmission, the acquisition apparatus may be equipped in its electronic unit with a wireless communication coupler 92 configured to transmit or even transmit and receive data in cooperation with a remote unit 94 (smartphone or remote server). BlueTooth™ or BlueTooth Low Emission™ protocols, and/or Wi-Fi may be used.

The case 11 may be housed next to the acquisition portion 1A, along the longitudinal direction X. For example, the case may be mounted on the thin sheet forming the sensor 5. The cover 8 is then dimensioned so as to accommodate the case and the acquisition portion.

The acquisition apparatus may have a length of between 50 cm and 1 m, or between 50 and 80 cm, or between 60 and 70 cm. The acquisition apparatus may have a width between 10 and 30 cm, or even between 15 and 25 cm, or even between 18 and 23 cm. The cover defines the exterior of the acquisition apparatus and has the same dimensions. The general shape of the acquisition apparatus may be rectangular, with a thickness between 0.4 cm and 2 cm, or even between 0.4 cm and 1 cm, or even around 5 mm. The mass of the acquisition apparatus may be lower than 500 g.

1. An acquisition apparatus for acquiring physiological data of a living being, configured to be installed in a bed with bed frame elements, the acquisition apparatus comprising an acquisition portion which comprises:
a sensor adapted to detect mechanical waves passing through the bedding,
a support device comprising at least one rigid panel, configured to support the sensor,
the support device being configured to rest on bed frame elements in a configuration of use of the acquisition apparatus in the bed, so that the support device is interposed between the bed frame elements and the sensor.

2. The acquisition apparatus according to claim 1, wherein the acquisition portion extends along a longitudinal direction and the support device conprises a plurality of rigid panels arranged next to eah other along the longitudinal axis of the acquisition portion, connected to each other by folding zone allowing folding of the support device.

3. The acquisition apparatus according to claim 2, wherein the plurality of the rigid panels are integrated into a flexible sheath, which extends along the longitudinal direction, wherein areas of the flexible sheath devoid of the plurality of the rigid panels form folding zones.

4. The acquisition apparatus according to claim 3, wherein the flexible sheath forms a plurality of housings, with the plurality of the rigid panels inserted into the housings.

5. The acquisition apparatus according to claim 4, wherein each of the plurality of housings receives a single one of the plurality of the rigid panels.

6. The acquisition apparatus according to claim 2, wherein each rigid panel has a width along a transverse axis orthogonal to the longitudinal axis of between 3 cm and 10 cm.

7. The acquisition apparatus according to claim 2, wherein the acquisition apparatus is configured to be folded into a storage configuration of the acquisition apparatus.

8. The acquisition apparatus according to claim 7, wherein the sensor comprises a pneumatic chamber, coupled to a pump and a discharge valve, the pneumatic chamber being configured to be inflated in the use configuration and to be deflated in the storage configuration.

9. The acquisition apparatus according to claim 1, wherein the sensor has a sensor width along a transverse direction and each rigid panel extends, along the transverse direction, over at least 80% of a sensor width.

10. The acquisition apparatus according to claim 1, further comprising a cover enveloping at least the acquisition portion.

11. The acquisition apparatus according to claim 10, wherein the cover is removable.

12. The acquisition apparatus according to claim 4, further comprising a cover enveloping at least the acquisition portion, wherein the cover comprises a top face, to be positioned against the mattress and a bottom face, to be positioned against the bed frame elements, the cover being configured to receive the sensor between the top face and the bottom face, and wherein the flexible sheath forms the bottom face of the cover.

13. The acquisition apparatus according to claim 1, further comprising at least one attachment, the at least one attachment being configured to be attached to bed frame elements.

14. The acquisition apparatus according to claim 1, wherein at least one of the at least one rigid panel is solid.

15. The acquisition apparatus according to claim 1, wherein at least one of the at least one rigid panel is apertured.

16. The acquisition apparatus according to claim 1, further comprising an electronic unit, configured to process raw data delivered by the sensor.

17. The acquisition apparatus according to claim 1, comprising a visual marker indicating to the user a direction of use of the acquisition apparatus, the visual marker and the support device each being on an opposite side of the sensor.

18. The acquisition apparatus according to claim 17, wherein the visual marker is a logo.

19. A kit for assembling an acquisition apparatus according to claim 1, the kit comprising the sensor and the support device.

20. An assembly comprising a bed frame element and an acquisition apparatus according to claim 1, wherein the bed frame element comprises two slats having a length that extends along a longitudinal direction and spaced apart by an inter-slat distance along a transverse direction, wherein a width of the sensor is greater than the inter-slat distance and/or each rigid panel extends along the transverse direction over a width greater than the inter-slat distance.
